(19) United States
(12) Patent Application Publication
(10) Pub. No.: US 2009/0053736 A1
(43) Pub. Date: Feb. 26, 2009

(54) Homogeneous Chemiluminescent Immunoassay for Analysis of Iron Metalloproteins

(76) Inventors: Phillip G. Mattingly, Third Lake, IL (US); Maciej Adamczyk, Gurnee, IL (US); Roy Jeffrey Brashear, Mundelein, IL (US)

Correspondence Address: Robert DeBerardine;D-377/AP6A-1, Abbott Laboratories, 100 Abbott Park Road, Abbott Park, IL, 60064-6008, US

(21) Appl. No.: 11/842,890

(22) Filed: Aug. 21, 2007

(51) Int. Cl. G01N 33/573

(52) U.S. Cl. 435/7.4

(57) ABSTRACT

The present invention relates to assays and kits for detecting or quantifying iron metalloprotein in test samples.

### RELATED APPLICATION INFORMATION

None.

### FIELD OF THE INVENTION

The present invention relates to assays and kits for detecting or quantifying an iron metalloprotein in a test sample.

### BACKGROUND OF THE INVENTION

One-third of all proteins are “metalloproteins”. Metalloproteins are chemical combinations of proteins with ions of metal such as iron, calcium, copper and zinc. Iron metalloproteins, for example, comprise combinations of proteins with ions of iron. The metal ions in metalloproteins are critical to the protein's function, structure, or stability. In fact, numerous essential biological functions require metal ions, and most of these metal ions functions involve metalloproteins.

Iron metalloproteins may be classified as heme-proteins or non-heme proteins, as described in Sykes, A. G., and Mauk, G., eds. Heme-Fe Proteins Advances in Inorganic Chemistry. Vol. 51: Academic Press, 2000 or Messerschmidt, A., et al., eds. Handbook of Metalloproteins, pages 3-864 (Wiley, 2004). As alluded to above, iron metalloproteins are involved in a number of important biological functions. For example, it is known that the heme-protein, myeloperoxidase is secreted by white blood cells. White blood cells are also known to generate hydrogen peroxide. In the presence of hydrogen peroxide, myeloperoxidase catalyzes the oxidation of chloride to hypochlorous acid (HOCl). HOCl is a potent cytotoxin for bacteria, viruses and fungi. The generation of HOCl by white blood cells plays a key role in host defenses against invading pathogens.

However, while iron metalloproteins play important roles in a number of biological functions, many iron metalloproteins are also known to cause oxidative injury resulting in tissue damage in disorders ranging from arthritis to ischemia reperfusion injury to cancer. For example, an elevated level of myeloperoxidase in subjects with cardiovascular disease has been associated with arterial inflammation. A number of studies have linked arterial inflammation with an increased risk of cardiovascular events. Decreased levels of frataxin are associated with Friedreich's ataxia, an autosomal cardio- and neurodegenerative disorder that affects 1 in 50,000 humans. Eosinophil peroxidase has been implicated in promoting oxidative tissue damage in a variety of inflammatory conditions, including asthma. Increased levels of lactoferrin are found in a number of inflammatory conditions, such as inflammatory bowel disease.

Therefore, there is a need in the art for new methods of detecting the amount of iron metalloproteins in a test sample.

### SUMMARY OF THE PRESENT INVENTION

In one embodiment, the present invention relates to a method of detecting an iron metalloprotein in a test sample. The method comprises the steps of:

a) adding an acridinium-9-carboxamide-antibody conjugate to a test sample, wherein the antibody specifically binds the iron metalloprotein;

b) generating in or providing to the test sample a source of hydrogen peroxide before or after the addition of an acridinium-9-carboxamide-antibody conjugate;

c) adding a basic solution to the test sample to generate a light signal; and

d) measuring the light generated to detect the iron metalloprotein.

The test sample used in the above-described method can be whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid or semen. The iron metalloprotein detected in the above-described method can be selected from the group consisting of: myeloperoxidase, ferritin, transferrin, lactoperoxidase, lactoferrin, ferredoxin, frataxin, divalent metal transporter 1, myoinositol oxygenase, rubrerythrin, thyroid peroxidase, methemoglobin and hemoglobin.

In the above-described method, the source of hydrogen peroxide can be provided by adding a buffer or a solution containing hydrogen peroxide. Alternatively, the hydrogen peroxide is generated by adding a hydrogen peroxide generating enzyme to the test sample.

In the above-described method, the acridinium-9-carboxamide-antibody conjugate is prepared from an acridinium-9-carboxamide having a structure according to formula I:

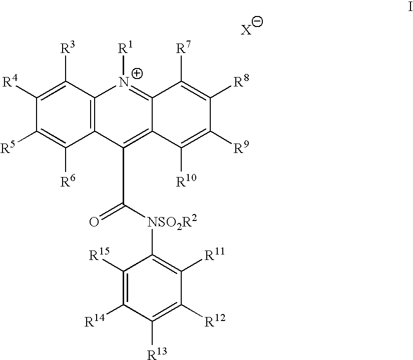

wherein R¹ and R² are each independently selected from the group consisting of: alkyl, alkenyl, alkynyl, aryl or aralkyl, sulfoalkyl, carboxyalkyl and oxoalkyl, and
wherein R³ through R¹⁵ are each independently selected from the group consisting of: hydrogen, alkyl, alkenyl, alkynyl, aryl or aralkyl, amino, amido, acyl, alkoxyl,
   hydroxyl, carboxyl, halogen, halide, nitro, cyano, sulfo, sulfoalkyl, carboxyalkyl and oxoalkyl; and
optionally, if present, X^{⊖} is an anion.

The antibody that can be used as part of the conjugate can be a polyclonal antibody, a monoclonal antibody, a chimeric antibody, a human antibody, a humanized antibody, a recombinant antibody, a single-chain Fv, an affinity maturated antibody, a single chain antibody, a single domain antibody, a Fab fragment, a F(ab′) fragment, a disulfide-linked Fv, an anti-idiotypic antibody and a functionally active epitope-binding fragment of any of the above.

Additionally, the above method further optionally comprises quantifying the amount of iron metalloprotein in the test sample by relating the amount of light generated in the test sample by comparison to a standard curve for said iron metalloprotein. Also, optionally, the standard curve can be generated from solutions of an iron metalloprotein of a known concentration.

In another embodiment, the present invention relates to a kit for use in detecting an iron metalloprotein in a test sample. The kit can comprise:
a. at least one acridinium-9-carboxamide;
b. at least one antibody that specifically binds the iron metalloprotein;
c. at least one basic solution;
d. a source of hydrogen peroxide; and
e. instructions for detecting an iron metalloprotein in a test sample.

The at least one acridinium-9-carboxamide in the above kit can have a structure according to formula I:

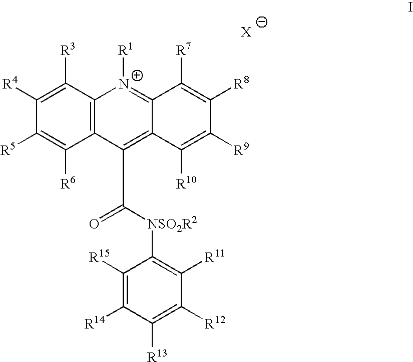

wherein R¹ and R² are each independently selected from the group consisting of: alkyl, alkenyl, alkynyl, aryl or aralkyl, sulfoalkyl, carboxyalkyl and oxoalkyl, and
wherein R³ through R¹⁵ are each independently selected from the group consisting of: hydrogen, alkyl, alkenyl, alkynyl, aryl or aralkyl, amino, amido, acyl, alkoxyl,
   hydroxyl, carboxyl, halogen, halide, nitro, cyano, sulfo, sulfoalkyl, carboxyalkyl and oxoalkyl; and
optionally, if present, X^{⊖} is an anion.

The source of hydrogen peroxide in the above-described kit can be a buffer or a solution containing hydrogen peroxide.

In another embodiment, the present invention relates to another kit for use in detecting

an iron metalloprotein in a test sample. The kit can comprise:
a. at least one acridinium-9-carboxamide;
b. at least one antibody that specifically binds the iron metalloprotein;
c. at least one basic solution;
d. a means of generating hydrogen peroxide in situ in the test sample; and
e. instructions for detecting an iron metalloprotein in a test sample.

The at least one acridinium-9-carboxamide in the above kit can have a structure according to formula I:

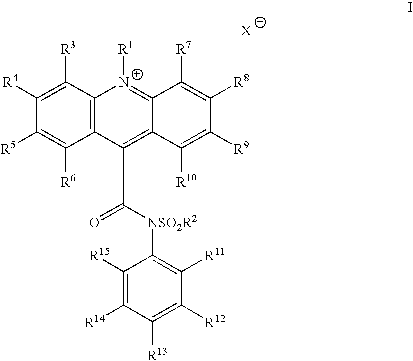

wherein R¹ and R² are each independently selected from the group consisting of: alkyl, alkenyl, alkynyl, aryl or aralkyl, sulfoalkyl, carboxyalkyl and oxoalkyl, and
wherein R³ through R¹⁵ are each independently selected from the group consisting of: hydrogen, alkyl, alkenyl, alkynyl, aryl or aralkyl, amino, amido, acyl, alkoxyl,
   hydroxyl, carboxyl, halogen, halide, nitro, cyano, sulfo, sulfoalkyl, carboxyalkyl and oxoalkyl; and
optionally, if present, X^{⊖} is an anion.

The above-described kit can further comprise instructions for generating hydrogen peroxide in situ in the test sample.

Additionally, the means for generating hydrogen peroxide in situ in the test sample contained in the kit can be at least one hydrogen peroxide generating enzyme. The at least one hydrogen peroxide generating enzyme can be selected from the group consisting of: (R)-6-hydroxynicotine oxidase, (S)-2-hydroxy acid oxidase, (S)-6-hydroxynicotine oxidase, 3-aci-nitropropanoate oxidase, 3-hydroxyanthranilate oxidase, 4-hydroxymandelate oxidase, 6-hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono-1,4-lactone oxidase, D-arabinono-1,4-lactone oxidase, D-aspartate oxidase, D-glutamate oxidase, D-glutamate(D-aspartate) oxidase, dihydrobenzophenanthridine oxidase, dihydroorotate oxidase, dihydrouracil oxidase, dimethylglycine oxidase, D-mannitol oxidase, ecdysone oxidase, ethanolamine oxidase, galactose oxidase, glucose oxidase, glutathione oxidase, glycerol-3-phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L-glutamate oxidase, L-gulonolactone oxidase, L-lysine 6-oxidase, L-lysine oxidase, long-chain-alcohol oxidase, L-pipecolate oxidase, L-sorbose oxidase, malate oxidase, methanethiol oxidase, monoamino acid oxidase, N⁶-methyl-lysine oxidase, N-acylhexosamine oxidase, AND(P)H oxidase, nitroalkane oxidase, N-methyl-L-amino-acid oxidase, nucleoside oxidase, oxalate oxidase, polyamine oxidase, polyphenol oxidase, polyvinyl-alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan α,β-oxidase, urate oxidase (uricase, uric acid oxidase), vanillyl-alcohol oxidase, xanthine oxidase, xylitol oxidase and combinations thereof.

### DETAILED DESCRIPTION OF THE PRESENT INVENTION

The present invention provides assays and kits for detecting or quantifying an iron metalloprotein in a test sample. Immunoassays for iron metalloprotein known in the art typically are heterogeneous assays that employ both a capture antibody on a solid support (e.g., microparticle, microplate, tube, and the like) and a detection antibody conjugated to a label (e.g., enzyme, chemiluminophore, and others such as are well known). The disadvantages of heterogeneous sandwich assays are well known, and include lack of reproducibility of the preparation of both the solid phase and the antibody-labeled solid phase, and the complex automation needed in automated immunoassays for manipulation of the solid support while performing the assay. Immunoassays for the iron metalloprotein myeloperoxidase further are complicated by the presence of autoantibodies in a large percentage of samples, which block the interaction of myeloperoxidase with the solid-phase and/or detection antibodies.

The invention described herein circumvents some of the disadvantages of the prior art assays and presents a novel and unexpected solution to the challenge of detecting or quantifying an iron metalloprotein in a test sample. In particular, the invention optionally can be employed in a homogeneous immunoassay format. The invention optionally uses only a single antibody conjugate. The invention optionally does not employ a solid support. The invention optionally reduces, minimizes, or eliminates the probability or actuality of auto-antibody interference in an iron metalloprotein immunoassay. Additional objects and advantages of the invention would be apparent from the further description provided herein.

A. Definitions

As used herein, the term “acyl” refers to a —C(O)Rₐ group where Rₐ is hydrogen, alkyl, cycloalkyl, cycloalkylalkyl, phenyl or phenylalkyl. Representative examples of acyl include, but are not limited to, formyl, acetyl, cylcohexylcarbonyl, cyclohexylmethylcarbonyl, benzoyl, benzylcarbonyl and the like.

As used herein, the term “alkenyl” means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

As used herein, the term “alkyl” means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

As used herein, the term “alkyl radical” means any of a series of univalent groups of the general formula CₙH2ₙ+1 derived from straight or branched chain hydrocarbons.

As used herein, the term “alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

As used herein, the term “alkynyl” means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

As used herein, the term “amido” refers to an amino group attached to the parent molecular moiety through a carbonyl group (wherein the term “carbonyl group” refers to a —C(O)— group).

As used herein, the term “amino” means —NR_{b}R_{c}, wherein R_{b} and R_{c} are independently selected from the group consisting of hydrogen, alkyl and alkylcarbonyl.

As used herein, the term “anion” refers to an anion of an inorganic or organic acid, such as, but not limited to, hydrochloric acid, hydrobromic acid, sulfuric acid, methane sulfonic acid, formic acid, acetic acid, oxalic acid, succinic acid, tartaric acid, mandelic acid, fumaric acid, lactic acid, citric acid, glutamic acid, aspartic acid, phosphate, trifluoromethansulfonic acid, trifluoroacetic acid and fluorosulfonic acid and any combinations thereof.

As used herein, “antibody” refers to immunoglobulin molecules and immunologically active portions of immunoglobulin (Ig) molecules, i.e., molecules that contain an antigen-binding site that specifically binds (immunoreacts with) an antigen. Such antibodies include, but are not limited to, polyclonal, monoclonal, chimeric, single chain, Fab, Fab′ and F(ab′)₂ fragments, etc. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species. In one embodiment, the antibody specifically binds an iron metalloproteinase.

As used herein, the term “aralkyl” means an aryl group appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

As used herein, the term “aryl” means a phenyl group, or a bicyclic or tricyclic fused ring system wherein one or more of the fused rings is a phenyl group. Bicyclic fused ring systems are exemplified by a phenyl group fused to a cycloalkenyl group, a cycloalkyl group, or another phenyl group. Tricyclic fused ring systems are exemplified by a bicyclic fused ring system fused to a cycloalkenyl group, a cycloalkyl group, as defined herein or another phenyl group. Representative examples of aryl include, but are not limited to, anthracenyl, azulenyl, fluorenyl, indanyl, indenyl, naphthyl, phenyl, and tetrahydronaphthyl. The aryl groups of the present invention can be optionally substituted with one-, two, three, four, or five substituents independently selected from the group consisting of alkoxy, alkyl, carboxyl, halo, and hydroxyl.

As used herein, the term “carboxy” or “carboxyl” refers to —CO₂H or —CO₂^{—}.

As used herein, the term “carboxyalkyl” refers to a —(CH₂)ₙCO₂^{—} or —(CH₂)ₙCO₂H group where n is from 1 to 10.

As used herein, the term “cyano” means a —CN group.

As used herein, the term “cycloalkenyl” refers to a non-aromatic cyclic or bicyclic ring system having from three to ten carbon atoms and one to three rings, wherein each five-membered ring has one double bond, each six-membered ring has one or two double bonds, each seven- and eight-membered ring has one to three double bonds, and each nine-to ten-membered ring has one to four double bonds. Representative examples of cycloalkenyl groups include cyclohexenyl, octahydronaphthalenyl, norbornylenyl, and the like. The cycloalkenyl groups can be optionally substituted with one, two, three, four, or five substituents independently selected from the group consisting of alkoxy, alkyl, carboxyl, halo, and hydroxyl.

As used herein, the term “cycloalkyl” refers to a saturated monocyclic, bicyclic, or tricyclic hydrocarbon ring system having three to twelve carbon atoms. Representative examples of cycloalkyl groups include cyclopropyl, cyclopentyl, bicyclo[3.1.1]heptyl, adamantyl, and the like. The cycloalkyl groups of the present invention can be optionally substituted with one, two, three, four, or five substituents independently selected from the group consisting of alkoxy, alkyl, carboxyl, halo, and hydroxyl.

As used herein, the term “cycloalkylalkyl” means a —R_{d}Rₑ group where R_{d} is an alkylene group and Rₑ is cycloalkyl group. A representative example of a cycloalkylalkyl group is cyclohexylmethyl and the like.

As used herein, the term “halogen” means a —Cl, —Br, —I or —F; the term “halide” means a binary compound, of which one part is a halogen atom and the other part is an element or radical that is less electronegative than the halogen, e.g., an alkyl radical.

As used herein, the term “hydroxyl” means an —OH group.

As used herein, the term “nitro” means a —NO₂ group.

As used herein, the term “oxoalkyl” refers to —(CH₂)ₙC(O)Rₐ, where Rₐ is hydrogen, alkyl, cycloalkyl, cycloalkylalkyl, phenyl or phenylalkyl and where n is from 1 to 10.

As used herein, the term “phenylalkyl” means an alkyl group which is substituted by a phenyl group.

As used herein, the term “sulfo” means a —SO₃H or —SO₃^{—} group.

As used herein, the term “sulfoalkyl” refers to a —(CH₂)ₙSO₃H or —(CH₂)ₙSO₃^{—} group where n is from 1 to 10.

As used herein, the term “test sample” generally refers to a biological material being tested for and/or suspected of containing an analyte of interest, such as an iron metalloprotein. The test sample may be derived from any biological source, such as, a physiological fluid, including, but not limited to, whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid, semen and so forth. Besides physiological fluids, other liquid samples may be used such as water, food products, and so forth, for the performance of environmental or food production assays. In addition, a solid material suspected of containing the analyte may be used as the test sample. The test sample may be used directly as obtained from the biological source or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release the analyte.

As used in this specification and the appended claims, the singular forms “a”, “an”, and “the” include plural references unless the context clearly dictates otherwise. Thus, for example, a reference to “the method” includes one or more methods, and/or steps of the type described herein and/or which will become apparent to those persons skilled in the art upon reading this disclosure and so forth.

B. Assay for Detecting or Quantifying Iron Metalloproteins

In general, the present invention relates to an assay for detecting or quantifying an iron metalloprotein in a test sample.

1. Test Sample

The assay or method of the present invention involves obtaining a test sample from a subject. A subject from which a test sample can be obtained is any vertebrate. Preferably, the vertebrate is a mammal. Examples of mammals include, but are not limited to, dogs, cats, rabbits, mice, rats, goats, sheep, cows, pigs, horses, non-human primates and humans. The test sample can be obtained from the subject using routine techniques known to those skilled in the art. Preferably, the test sample contains one or more iron metalloproteins, such as, but not limited to, myeloperoxidase, ferritin, transferrin, lactoperoxidase, lactoferrin, ferredoxin, frataxin, divalent metal transporter 1, myoinositol oxygenase, rubrerythrin, thyroid peroxidase, methemoglobin, hemoglobin, or any combinations thereof. Optionally, the test sample contains cells which produce or secrete one or more iron metalloproteins, such as, but not limited to, myeloperoxidase, ferritin, transferrin, lactoperoxidase, lactoferrin, ferredoxin, frataxin, divalent metal transporter 1, myoinositol oxygenase, rubrerythrin, thyroid peroxidase, methemoglobin, hemoglobin, or any combinations thereof.

2. Acridinium-9-carboxamide

After the test sample is obtained from a subject, at least one acridinium carboxamide-antibody conjugate is added to the test sample. Preferably, the acridinium carboxamide-antibody conjugate is prepared from an acridinium-9-carboxamide, including optionally an acridinium-9-carboxamide having a structure according to formula I shown below:

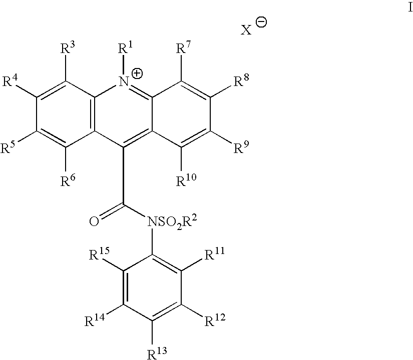

wherein R¹ and R² are each independently selected from the group consisting of: alkyl, alkenyl, alkynyl, aryl or aralkyl, sulfoalkyl, carboxyalkyl and oxoalkyl, and
wherein R³ through R¹⁵ are each independently selected from the group consisting of: hydrogen, alkyl, alkenyl, alkynyl, aryl or aralkyl, amino, amido, acyl, alkoxyl,
   hydroxyl, carboxyl, halogen, halide, nitro, cyano, sulfo, sulfoalkyl, carboxyalkyl and oxoalkyl;
and further wherein any of the alkyl, alkenyl, alkynyl, aryl or aralkyl may contain one or more heteroatoms; and
optionally, if present, X^{⊖} is an anion.

Methods for preparing acridinium 9-carboxamides are described in Mattingly, P. G. J. Biolumin. Chemilumin., 6, 107-14 (1991); Adamczyk, M.; Chen, Y.-Y.; Mattingly, P. G.; Pan, Y. J. Org. Chem., 63, 5636-5639 (1998); Adamczyk, M.; Chen, Y.-Y.; Mattingly, P. G.; Moore, J. A.; Shreder, K. Tetrahedron, 55, 10899-10914 (1999); Adamczyk, M.; Mattingly, P. G.; Moore, J. A.; Pan, Y. Org. Lett., 1, 779-781 (1999); Adamczyk, M.; Chen, Y.-Y.; Fishpaugh, J. R.; Mattingly, P. G.; Pan, Y.; Shreder, K.; Yu, Z. Bioconjugate Chem., 11, 714-724 (2000); Mattingly, P. G.; Adamczyk, M. In Luminescence Biotechnology: Instruments and Applications; Dyke, K. V. Ed.; CRC Press: Boca Raton, pp. 77-105 (2002); Adamczyk, M.; Mattingly, P. G.; Moore, J. A.; Pan, Y. Org. Lett., 5, 3779-3782 (2003); and U.S. Pat. Nos. 5,468,646, 5,543,524, and 5,783,699 (each incorporated herein by reference in their entireties for their teachings regarding same).

3. Antibody

The antibody used in the conjugate can be any antibody known in the art that preferably is directed against an iron metalloprotein. For example, the antibody can be a polyclonal or monoclonal antibody, a chimeric antibody, a humanized antibody or a human antibody. Various procedures known within the art may be used for the production of polyclonal antibodies, monoclonal antibodies, chimeric antibodies, humanized antibodies, human antibodies, etc. Some of these methods and antibodies are discussed in more detail below.

For the production of polyclonal antibodies, various suitable host animals (e.g., rabbit, goat, mouse or any other mammal) may be immunized by one or more injections with a native protein, a synthetic variant thereof, or a derivative thereof. An appropriate immunogenic preparation can contain, for example, the naturally occurring immunogenic protein, a chemically synthesized polypeptide representing the immunogenic protein, or a recombinantly expressed immunogenic protein. Furthermore, a protein may be conjugated to a second protein known to be immunogenic in the mammal being immunized. Examples of such immunogenic proteins include, but are not limited to, keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface-active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants that can be employed include MPL-TDM adjuvant (monophosphoryl Lipid A, synthetic trehalose dicorynomycolate).

The polyclonal antibody molecules directed against an immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is well known to those skilled in the art.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include a protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium (DMEM) and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368:812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Chimeric monoclonal antibodies can be produced by recombinant DNA techniques known in the art. For example, a gene encoding the constant region of a non-human antibody molecule is substituted with a gene encoding a human constant region (See, for example, PCT Patent Publication PCT/US86/02269, European Patent Application 184,187 or European Patent Application 171,496).

Humanized antibodies are antibodies that are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann, et al., Nature, 332:323-327 (1988); Verhoeyen, et al., Science, 239:1534-1536 (1988)), by substituting rodent CDRs or CDR sequences for the corresponding sequences of a human antibody (See also U.S. Pat. No. 5,225,539). In some instances, Fv framework residues of the human immunoglobulin are replaced by corresponding non-human residues. Humanized antibodies can also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann, et al., Nature, 332:323-327 (1988); and Presta, Curr. Op. Struct. Biol., 2:593-596 (1992)).

Fully human antibodies refer to antibody molecules in which essentially the entire sequences of both the light chain and the heavy chain, including the CDRs, arise from human genes. Such antibodies are termed “human antibodies” or “fully human antibodies” herein. Human monoclonal antibodies can be prepared by the trioma technique; the human B-cell hybridoma technique (See, Kozbor, et al., Immunol Today 4: 72 (1983)) and the EBV hybridoma technique to produce human monoclonal antibodies (See, Cole, et al., 1985 In: Monoclonal Antibodies and Cancer Therapy, Alan R. Liss, Inc., pp. 77-96). Human monoclonal antibodies may be utilized in the practice of the present invention and may be produced by using human hybridomas (See, Cote, et al., Proc Natl Acad Sci USA 80: 2026-2030 (1983)) or by transforming human B-cells with Epstein Barr Virus in vitro (See, Cole, et al., 1985 In: Monoclonal Antibodies and Cancer Therapy, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10:779-783 (1992)); Lonberg et al. (Nature 368:856-859 (1994)); Morrison (Nature 368:812-13 (1994)); Fishwild et al., (Nature Biotechnology, 14:845-51 (1996)); Neuberger (Nature Biotechnology, 14:826 (1996)); and Lonberg and Huszar (Intern. Rev. Immunol. 13:65-93 (1995)).

Human antibodies may additionally be produced using transgenic nonhuman animals which are modified so as to produce fully human antibodies rather than the animal's endogenous antibodies in response to challenge by an antigen (See, PCT International Application WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse® as disclosed in PCT International Applications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

Single-chain antibodies specific to an antigenic protein (See, e.g., U.S. Pat. No. 4,946,778) can also be used. In addition, methods can be adapted for the construction of Fab expression libraries (See, e.g., Huse, et al., Science 246:1275-1281 (1989)) to allow rapid and effective identification of monoclonal Fab fragments with the desired specificity for a protein or derivatives, fragments, analogs or homologs thereof. Antibody fragments that contain the idiotypes to a protein antigen may be produced by techniques known in the art including, but not limited to: (i) an F(ab′)₂ fragment produced by pepsin digestion of an antibody molecule; (ii) an Fab fragment generated by reducing the disulfide bridges of an F(ab′)₂ fragment; (iii) an Fab fragment generated by the treatment of the antibody molecule with papain and a reducing agent and (iv) Fv fragments.

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in PCT International Application WO 93/08829 and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in PCT International Application WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies can be prepared as full-length antibodies or antibody fragments (e.g., F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers (See, Kostelny et al., J. Immunol. 148:1547-1553 (1992)). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies can also be used. For example, trispecific antibodies can be prepared (See, Tutt et al., J. Immunol. 147:60 (1991))

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g., CD2, CD3, CD28, or B7), or Fc receptors for IgG (FcΓR), such as FcΓRI (CD64), FcΓRII (CD32) and FcΓRIII (CD 16), so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Additionally, the antibody can be any antibody directed against an iron metalloprotein that is commercially available and/or which has been described in the literature. For example, there are a number of commercial ELISA kits for myeloperoxidase that employ antibodies that might be used in an assay according to the invention, e.g., antibodies contained in the Invitrogen ZEN™ Myeloperoxidase (MPO) ELISA kit, or the PrognostiX CardioMPO™ kit.

4. Antibody Conjugate

The antibody selected for use in the conjugate can be linked, coupled, etc. using routine techniques known in the art. Examples of techniques that can be used include, but are not limited to those described in Hermanson, Greg T. Bioconjugate Techniques. San Diego: Academic Press, (1996), Niemeyer, Christof M., ed. Bioconjugation Protocols: Strategies and Methods. Totowa, N.J.: Humana Press (2004).

5. Hydrogen Peroxide Source

In one embodiment of the present invention, hydrogen peroxide is generated in situ in the test sample or provided or supplied to the test sample before the addition of the above-described acridinium-9-carboxamide-antibody conjugate. In a second embodiment of the present invention, the hydrogen peroxide is generated in situ in the test ample or provided or supplied to the test sample simultaneously with the above-described acridinium-9-carboxamide-antibody conjugate. In a third embodiment, hydrogen peroxide is generated in situ or provided or supplied to the test sample after the above-described acridinium-9-carboxamide-conjugate is added to the test sample.

As mentioned above, hydrogen peroxide can be generated in situ in the test sample. Hydrogen peroxide can be generated in situ in a number of ways. For example, a number of enzymes are known in the art that are capable of generating hydrogen peroxide (which is also referred to herein as a hydrogen peroxide generating enzyme). Such enzymes are listed below in Table 1.

TABLE 1

| | IUBMB ENZYME | PREFERRED |
|---|---|---|
| ACCEPTED COMMON NAME | NOMENCLATURE | SUBSTRATE |
| (R)-6-hydroxynicotine oxidase | EC 1.5.3.6 | (R)-6-hydroxynicotine |
| (S)-2-hydroxy acid oxidase | EC 1.1.3.15 | S)-2-hydroxy acid |
| (S)-6-hydroxynicotine oxidase | EC 1.5.3.5 | (S)-6-hydroxynicotine |
| 3-aci-nitropropanoate oxidase | EC 1.7.3.5 | 3-aci-nitropropanoate |
| 3-hydroxyanthranilate oxidase | EC 1.10.3.5 | 3-hydroxyanthranilate |
| 4-hydroxymandelate oxidase | EC 1.1.3.19 | (S)-2-hydroxy-2-(4- |
| | | hydroxyphenyl)acetate |
| 6-hydroxynicotinate dehydrogenase | EC 1.17.3.3 | 6-hydroxynicotinate |
| abscisic-aldehyde oxidase | EC 1.2.3.14 | abscisic aldehyde |
| acyl-CoA oxidase | EC 1.3.3.6 | acyl-CoA |
| alcohol oxidase | EC 1.1.3.13 | primary alcohol |
| aldehyde oxidase | EC 1.2.3.1 | Aldehyde |
| amine oxidase | | |
| amine oxidase (copper-containing) | EC 1.4.3.6 | primary monoamines, |
| | | diamines and histamine |
| amine oxidase (flavin-containing) | EC 1.4.3.4 | primary amine |
| aryl-alcohol oxidase | EC 1.1.3.7 | aromatic primary alcohol |
| | | (2-naphthyl)methanol |
| | | 3-methoxybenzyl alcohol |
| aryl-aldehyde oxidase | EC 1.2.3.9 | aromatic aldehyde |
| catechol oxidase | EC 1.1.3.14 | Catechol |
| cholesterol oxidase | EC 1.1.3.6 | Cholesterol |
| choline oxidase | EC 1.1.3.17 | Choline |
| columbamine oxidase | EC 1.21.3.2 | Columbamine |
| cyclohexylamine oxidase | EC 1.4.3.12 | Cyclohexylamine |
| cytochrome c oxidase | EC 1.9.3.1 | |
| D-amino-acid oxidase | EC 1.4.3.3 | D-amino acid |
| D-arabinono-1,4-lactone oxidase | EC 1.1.3.37 | D-arabinono-1,4-lactone |
| D-arabinono-1,4-lactone oxidase | EC 1.1.3.37 | D-arabinono-1,4-lactone |
| D-aspartate oxidase | EC 1.4.3.1 | D-aspartate |
| D-glutamate oxidase | EC 1.4.3.7 | D-glutamate |
| D-glutamate(D-aspartate) oxidase | EC 1.4.3.15 | D-glutamate |
| dihydrobenzophenanthridine | EC 1.5.3.12 | dihydrosanguinarine |
| oxidase | | |
| dihydroorotate oxidase | EC 1.3.3.1 | (S)-dihydroorotate |
| dihydrouracil oxidase | EC 1.3.3.7 | 5,6-dihydrouracil |
| dimethylglycine oxidase | EC 1.5.3.10 | N,N-dimethylglycine |
| D-mannitol oxidase | EC 1.1.3.40 | Mannitol |
| ecdysone oxidase | EC 1.1.3.16 | Ecdysone |
| ethanolamine oxidase | EC 1.4.3.8 | Ethanolamine |
| galactose oxidase | EC 1.1.3.9 | D-galactose |
| glucose oxidase | EC 1.1.3.4 | B-D-glucose |
| glutathione oxidase | EC 1.8.3.3 | Glutathione |
| glycerol-3-phosphate oxidase | EC 1.1.3.21 | sn-glycerol 3-phosphate |
| glycine oxidase | EC 1.4.3.19 | Glycine |
| glyoxylate oxidase | EC 1.2.3.5 | Glyoxylate |
| hexose oxidase | EC 1.1.3.5 | D-glucose, |
| | | D-galactose |
| | | D-mannose |
| | | maltose |
| | | lactose |
| | | cellobiose |
| hydroxyphytanate oxidase | EC 1.1.3.27 | L-2-hydroxyphytanate |
| indole-3-acetaldehyde oxidase | EC 1.2.3.7 | (indol-3-yl)acetaldehyde |
| lactic acid oxidase | | lactic acid |
| L-amino-acid oxidase | EC 1.4.3.2 | L-amino acid |
| L-aspartate oxidase | EC 1.4.3.16 | L-aspartate |
| L-galactonolactone oxidase | EC 1.3.3.12 | L-galactono-1,4-lactone |
| L-glutamate oxidase | EC 1.4.3.11 | L-glutamate |
| L-gulonolactone oxidase | EC 1.1.3.8 | L-gulono-1,4-lactone |
| L-lysine 6-oxidase | EC 1.4.3.20 | L-lysine |
| L-lysine oxidase | EC 1.4.3.14 | L-lysine |
| long-chain-alcohol oxidase | EC 1.1.3.20 | long-chain-alcohol |
| L-pipecolate oxidase | EC 1.5.3.7 | L-pipecolate |
| L-sorbose oxidase | EC 1.1.3.11 | L-sorbose |
| malate oxidase | EC 1.1.3.3 | (S)-malate |
| methanethiol oxidase | EC 1.8.3.4 | Methanethiol |
| monoamino acid oxidase | | |
| N⁶-methyl-lysine oxidase | EC 1.5.3.4 | 6-N-methyl-L-lysine |
| N-acylhexosamine oxidase | EC 1.1.3.29 | N-acetyl-D-glucosamine |
| | | N-glycolylglucosamine |
| | | N-acetylgalactosamine |
| | | N-acetylmannosamine. |
| NAD(P)H oxidase | EC 1.6.3.1 | NAD(P)H |
| nitroalkane oxidase | EC 1.7.3.1 | Nitroalkane |
| N-methyl-L-amino-acid oxidase | EC 1.5.3.2 | N-methyl-L-amino acid |
| nucleoside oxidase | EC 1.1.3.39 | Adenosine |
| oxalate oxidase | EC 1.2.3.4 | Oxalate |
| polyamine oxidase | EC 1.5.3.11 | 1-N-acetylspermine |
| polyphenol oxidase | EC 1.14.18.1 | |
| polyvinyl-alcohol oxidase | EC 1.1.3.30 | polyvinyl alcohol |
| prenylcysteine oxidase | EC 1.8.3.5 | S-prenyl-L-cysteine |
| protein-lysine 6-oxidase | EC 1.4.3.13 | peptidyl-L-lysyl-peptide |
| putrescine oxidase | EC 1.4.3.10 | butane-1,4-diamine |
| pyranose oxidase | EC 1.1.3.10 | D-glucose |
| | | D-xylose |
| | | L-sorbose |
| | | D-glucono-1,5-lactone |
| pyridoxal 5′-phosphate synthase | EC 1.4.3.5 | pyridoxamine 5′- |
| | | phosphate |
| pyridoxine 4-oxidase | EC 1.1.3.12 | Pyridoxine |
| pyrroloquinoline-quinone synthase | EC 1.3.3.11 | 6-(2-amino-2- |
| | | carboxyethyl)-7,8-dioxo- |
| | | 1,2,3,4,5,6,7,8- |
| | | octahydroquinoline-2,4- |
| | | dicarboxylate |
| pyruvate oxidase | EC 1.2.3.3 | Pyruvate |
| pyruvate oxidase (CoA-acetylating) | EC 1.2.3.6 | Pyruvate |
| reticuline oxidase | EC 1.21.3.3 | Reticuline |
| retinal oxidase | EC 1.2.3.11 | Retinal |
| rifamycin-B oxidase | EC 1.10.3.6 | rifamycin-B |
| sarcosine oxidase | EC 1.5.3.1 | Sarcosine |
| secondary-alcohol oxidase | EC 1.1.3.18 | secondary alcohol |
| sulfite oxidase | EC 1.8.3.1 | Sulfite |
| superoxide dismutase | EC 1.15.1.1 | Superoxide |
| superoxide reductase | EC 1.15.1.2 | Superoxide |
| tetrahydroberberine oxidase | EC 1.3.3.8 | (S)-tetrahydroberberine |
| thiamine oxidase | EC 1.1.3.23 | Thiamine |
| tryptophan α,β-oxidase | EC 1.3.3.10 | L-tryptophan |
| urate oxidase (uricase, uric acid | EC 1.7.3.3 | uric acid |
| oxidase) | | |
| vanillyl-alcohol oxidase | EC 1.1.3.38 | vanillyl alcohol |
| xanthine oxidase | EC 1.17.3.2 | Xanthine |
| xylitol oxidase | EC 1.1.3.41 | Xylitol |

One or more of the above-described enzymes can be added to the test sample in an amount sufficient to allow for the generation of hydrogen peroxide in situ in the test sample. The amount of one or more of the above enzymes to be added to the test sample can be readily determined by one skilled in the art.

Hydrogen peroxide can also be generated electrochemically in situ as shown in Agladze, G. R.; Tsurtsumia, G. S.; Jung, B. I.; Kim, J. S.; Gorelishvili, G. J. Applied Electrochem., 37, 375-383 (2007); Qiang, Z.; Chang, J.-H.; Huang, C.-P. Water Research, 36, 85-94 (2002), for example. Hydrogen peroxide can also be generated photochemically in situ, e.g., Draper, W. M.; Crosby, D. G. Archives of Environmental Contamination and Toxicology, 12, 121-126 (1983).

Alternatively, a source of hydrogen peroxide can be supplied to or provided in the test sample. For example, the source of the hydrogen peroxide can be one or more buffers or other solutions that are known to contain hydrogen peroxide. Such buffers or other solutions are simply added to the test sample. Alternatively, another source of hydrogen peroxide can simply be a solution containing hydrogen peroxide.

The amount of hydrogen peroxide generated in situ in the test sample or provided in or supplied to the test sample can be readily determined by one skilled in the art depending on the concentration of the specific iron metalloprotein to be detected or determined pursuant to the assay of the present invention. For example, the amount of hydrogen peroxide that can be generated in situ or provided in or supplied to the test sample is from about 0.0001 micromolar to about 200 micromolar.

As demonstrated by the above, the timing and order in which the acridinium-9-carboxamide-antibody conjugate and the hydrogen peroxide provided in or supplied to or generated in situ in the test sample is not critical provided that they are added, provided, supplied or generated in situ prior to the addition of at least one basic solution, which will be discussed in more detail below.

After the addition of the acridinium-9-carboxamide-antibody conjugate and the hydrogen peroxide to the test sample, at least one basic solution is added to the test sample in order to generate a detectable signal, namely, a chemiluminescent signal. The basic solution is the same basic solution discussed previously herein, namely, a solution that contains at least one base and that has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate and calcium bicarbonate. The amount of basic solution added to the test sample depends on the concentration of the basic solution used in the assay. Based on the concentration of the basic solution used, one skilled in the art could easily determine the amount of basic solution to be used in the method. Chemiluminescent signals generated can be detected using routine techniques known to those skilled in the art.

6. Signal Generation

Thus, in the assay of the present invention, the chemiluminescent signal generated after the addition of a basic solution, indicates the presence of an iron metalloprotein. The amount of the iron metalloprotein in the test sample can be quantified based on the intensity of the signal generated. Specifically, the amount of iron metalloprotein contained in a test sample is inversely proportional to the intensity of the signal generated. For example, in some instances, a high signal intensity may be generated by the lowest concentration of iron metalloprotein in the test sample (in this instance, the amount of iron metalloprotein in the test sample is inversely proportional to the amount of signal generated). Specifically, the amount of iron metalloprotein present can be quantified based on comparing the amount of light generated to a standard curve for the iron metalloprotein or by comparison to a reference standard. The standard curve can be generated using serial dilutions or solutions of the iron metalloprotein of known concentration, by mass spectroscopy, gravimetrically and by other techniques known in the art.

C. Kit for Detecting or Quantifying Iron Metalloproteins

In another embodiment, the present invention relates to a kit for determining or detecting an iron metalloprotein in a test sample. In one aspect, the kit can contain at least one acridinium-9-carboxamide having the structure according to formula I:

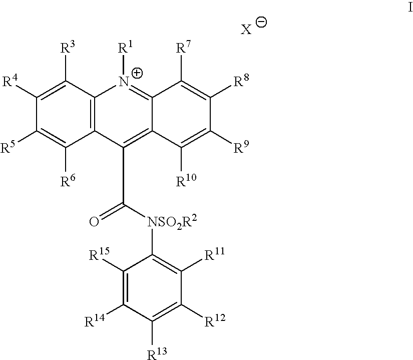

wherein R¹ and R² are each independently selected from the group consisting of: alkyl, alkenyl, alkynyl, aryl or aralkyl, sulfoalkyl, carboxyalkyl and oxoalkyl, and
wherein R³ through R¹⁵ are each independently selected from the group consisting of: hydrogen, alkyl, alkenyl, alkynyl, aryl or aralkyl, amino, amido, acyl, alkoxyl,
   hydroxyl, carboxyl, halogen, halide, nitro, cyano, sulfo, sulfoalkyl, carboxyalkyl and oxoalkyl;
and further wherein any of the alkyl, alkenyl, alkynyl, aryl or aralkyl may contain one or more heteroatoms; and
optionally, if present, X^{⊖} is an anion.

Additionally, the kit can also contain at least one antibody. The acridinium-9-carboxamide and antibody can each be provided separately in the kit with instructions describing how to prepare the acridinium-9-carboxamide-antibody conjugate. Alternatively, a preformed or premade acridinium-9-carboxamide-antibody conjugate can be included in the kit.

Moreover, the kit can also contain a source of hydrogen peroxide, such as one or more buffers or one or more solutions containing hydrogen peroxide. Furthermore, the kit can contain also at least one basic solution.

In yet another embodiment, the present invention relates to another kit for determining or detecting haloperoxidase activity in a test sample. In one aspect, the kit can contain at least one acridinium-9-carboxamide having a structure according to formula I:

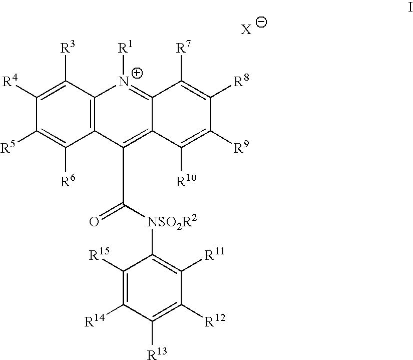

wherein R¹ and R² are each independently selected from the group consisting of: alkyl, alkenyl, alkynyl, aryl or aralkyl, sulfoalkyl, carboxyalkyl and oxoalkyl, and
wherein R³ through R¹⁵ are each independently selected from the group consisting of: hydrogen, alkyl, alkenyl, alkynyl, aryl or aralkyl, amino, amido, acyl, alkoxyl,
   hydroxyl, carboxyl, halogen, halide, nitro, cyano, sulfo, sulfoalkyl, carboxyalkyl and oxoalkyl;
and further wherein any of the alkyl, alkenyl, alkynyl, aryl or aralkyl may contain one or more heteroatoms; and
optionally, if present, X^{⊖} is an anion.

Additionally, the kit can also contain at least one antibody. The acridinium-9-carboxamide and antibody can each be provided separately in the kit with instructions describing how to prepare the acridinium-9-carboxamide-antibody conjugate. Alternatively, a preformed or premade acridinium-9-carboxamide-antibody conjugate can be included in the kit.

The kit can also contain at least one basic solution. Additionally, the kit can also contain a means of generating hydrogen peroxide in situ in the test sample. A means for generating hydrogen peroxide in situ in the test sample can include adding at least one hydrogen peroxide generating enzyme. A hydrogen peroxide generating enzyme that can be used can be selected from the group consisting of: (R)-6-hydroxynicotine oxidase, (S)-2-hydroxy acid oxidase, (S)-6-hydroxynicotine oxidase, 3-aci-nitropropanoate oxidase, 3-hydroxyanthranilate oxidase, 4-hydroxymandelate oxidase, 6-hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono-1,4-lactone oxidase, D-arabinono-1,4-lactone oxidase, D-aspartate oxidase, D-glutamate oxidase, D-glutamate(D-aspartate) oxidase, dihydrobenzophenanthridine oxidase, dihydroorotate oxidase, dihydrouracil oxidase, dimethylglycine oxidase, D-mannitol oxidase, ecdysone oxidase, ethanolamine oxidase, galactose oxidase, glucose oxidase, glutathione oxidase, glycerol-3-phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L-glutamate oxidase, L-gulonolactone oxidase, L-lysine 6-oxidase, L-lysine oxidase, long-chain-alcohol oxidase, L-pipecolate oxidase, L-sorbose oxidase, malate oxidase, methanethiol oxidase, monoamino acid oxidase, N⁶-methyl-lysine oxidase, N-acylhexosamine oxidase, AND(P)H oxidase, nitroalkane oxidase, N-methyl-L-amino-acid oxidase, nucleoside oxidase, oxalate oxidase, polyamine oxidase, polyphenol oxidase, polyvinyl-alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan α,β-oxidase, urate oxidase (uricase, uric acid oxidase), vanillyl-alcohol oxidase, xanthine oxidase, xylitol oxidase and combinations thereof.

D. Exemplary Formats

Exemplary formats of how the assay of the present invention can be performed are now provided.

Exemplary Format 1. Detection Conjugate.

The detection antibody is dissolved in a conjugation buffer (100 mM sodium phosphate, 150 mM NaCl, pH 8.0) to give a concentration of 1-10 mg/mL (6.25-62.5 μM). An acridinium-9-carboxamide labeling reagent is prepared in N,N-dimethylformamide (DMF) at a concentration of 1-50 mM. The selected antibody is treated with the acridinium-labeling reagent in a molar excess of 1-35 fold for 3-14 hours at ambient temperature in the dark. Afterwards, the acridinium-9-carboxamide-antibody conjugate solution is dialyzed at ambient temperature over 20 hours using a 10 kilodalton molecular weight cutoff membrane against three volumes (1000× conjugate solution volume) of a dialysis buffer consisting of 10 mM phosphate buffered saline (PBS) containing 0.1% CHAPS (3-[(3-cholamidopropyl)dimethylammonio]-1-propanesulfonate).

The acridinium-9-carboxamide-antibody conjugate is optionally purified by high performance liquid chromatography (HPLC) using a size-exclusion column. Thus, the acridinium-9-carboxamide-antibody conjugate solution (6 mL) is injected on a Waters HPLC (Milford, Mass.) consisting of a Waters 600 Controller and Waters 2487 Dual Wavelength Absorbance Detector and equipped with a TosoHaas (Montgomeryville, Pa.) G3000SW column. The conjugate is eluted at 4 mL/minute with a buffer consisting of 0.1% CHAPS/10 mM PBS, pH 6.3. The eluent is monitored by UV absorbance at 280/369 nm.

Collected fractions containing the acridinium-9-carboxamide-antibody conjugate may be pooled based on the incorporation ratio (IR) of the acridinium-9-carboxamide label to the protein calculated from the UV absorbance at 280/369 nm according to the formula:

IR=A369/ε369/([A280−(A369/4.1)]/ε280)

where A280 and A369 are absorbance values obtained from the UV-visible spectrum of the conjugate; 4.1 is the ˜A369/A280 ratio for an acridinium-9-carboxamide label; ε280 is the extinction coefficient for an antibody at 280 nm (i.e., for IgG mAb ε280=210,000 M⁻¹ cm⁻¹); and ε369 is the extinction coefficient for an acridinium-9-carboxamide label at 369 nm. The average incorporation ratio for pooled fractions can range from 0.4-0.8× the molar excess of the acridinium-9-carboxamide labeling reagent used.

Exemplary Format 2. Effect of acridinium-9-carboxamide/Antibody Incorporation Ratio (IR) and acridinium-9-carboxamide/Antibody Concentration on the Dose/Response to myeloperoxidase.

A series of detection conjugates are prepared according to Exemplary format 1 using an antibody specific for human myeloperoxidase and acridinium, 9-[[[(4-methylphenyl)sulfonyl](3-sulfopropyl)amino]carbonyl]-10-[10-oxo-10-(pentafluorophenoxy)decyl]-, inner salt (compound 1 from Example 1, described infra) with IR values of 1, 2, 4, 8, and 16. The conjugates are serially diluted to the range covering 0.005-50 nM in 0.1% CHAPS/10 mM PBS, pH 6.3.

Human neutrophil myeloperoxidase (Product Number 16-14-130000, Athens Research Technologies, Athens, Ga.) is diluted in PBS (pH 7.2) containing methionine (1 mM) to give solutions of 2900, 1450, 725, 362.50, 181.25, 90.63, 45.31, 22.66, 11.33, and 0.00 ng/mL.

Each conjugate dilution and each myeloperoxidase solution are mixed 1:1 and allowed to equilibrate for 1 hour at 28° C.

Each solution containing the detection conjugate and myeloperoxidase (20 μL) is then arrayed on a low-protein binding microplate in quadruplicate. The microplate is placed in a microplate luminometer (Mithras LB-940, BERTHOLD TECHNOLOGIES U.S.A. LLC, Oak Ridge, Tenn.) at 28° C. Well by well, a triggering solution (100 μL) (0.18N NaOH, 0.7% H₂O₂, 1% Triton X-100, 0.05% diethylenetriaminepentacetic acid) is added and the chemiluminescent signal recorded for 2 s.

The chemiluminescent response versus myeloperoxidase concentration is plotted for each concentration of each detection conjugate.

Alternatively, the normalized chemiluminescence response versus myeloperoxidase concentration is plotted for each concentration of each detection conjugate.

Exemplary Format 3. Effect of acridinium-9-carboxamide/Antibody Incorporation Ratio (IR) and acridinium-9-carboxamide/Antibody Concentration on the Dose/Response to myeloperoxidase.

Exemplary format 2 is done using acridinium, 9-[[[4-[(2,5-dioxo-1-pyrrolidinyl)oxy]-4-oxobutyl][(4-methylphenyl)sulfonyl]amino]carbonyl]-10-(3-sulfopropyl), inner salt (compound 2 from Example 1).

Exemplary Format 4. Effect of acridinium-9-carboxamide/Antibody Incorporation Ratio (IR) and acridinium-9-carboxamide/Antibody Concentration on the Dose/Response to myeloperoxidase.

Exemplary format 2 is done using acridinium, 9-[[[[4-[4-pentafluorophenoxy]-4-oxobutyl]phenyl]sulfonyl](3-sulfopropyl)amino]carbonyl]-10-(3-sulfopropyl), inner salt (compound 3 from Example 1).

Exemplary Format 5. Effect of acridinium-9-carboxamide/Antibody Incorporation Ratio (IR) and acridinium-9-carboxamide/Antibody Concentration on the Dose/Response to myeloperoxidase.

Exemplary format 2 is done using acridinium, 9-[[[[4-[4-[(2,5-dioxo-1-pyrrolidinyl)oxy]-4-oxobutyl]phenyl]sulfonyl](3-sulfopropyl)amino]carbonyl]-10-(3-sulfopropyl), inner salt (compound 4 from Example 1).

Exemplary Format 6. Effect of acridinium-9-carboxamide/Antibody Incorporation Ratio (IR) and acridinium-9-carboxamide/Antibody Concentration on the Dose/Response to thyroperoxidase.

Exemplary format 2 is done using an antibody specific for human thyroperoxidase.

Exemplary Format 7.

Exemplary format 2 is done using an antibody specific for human thyroperoxidase.

Exemplary Format 8.

Exemplary format 4 is done using an antibody specific for human thyroperoxidase.

Exemplary Format 9.

Exemplary format 5 is done using an antibody specific for human thyroperoxidase.

Exemplary Format 10.

Exemplary format 6 is done using an antibody specific for human thyroperoxidase.

Exemplary Format 11. Effect of acridinium-9-carboxamide/Antibody Incorporation Ratio (IR) and acridinium-9-carboxamide/Antibody Concentration on the Dose/Response to eosinophil peroxidase.

Exemplary formats 2 through 6 are done using an antibody specific for human eosinophil peroxidase.

Of course, it goes without saying that any of the exemplary formats herein, and any assay or kit according to the invention can be adapted or optimized for use in automated and semi-automated systems (including those in which there is a solid phase comprising a microparticle), as described, e.g., in U.S. Pat. Nos. 5,089,424 and 5,006,309, and as, e.g., commercially marketed by Abbott Laboratories (Abbott Park, Ill.) including but not limited to Abbott's ARCHITECT®, AxSYM, IMX, PRISM, and Quantum II platforms, as well as other platforms.

Additionally, the assays and kits of the present invention optionally can be adapted or optimized for point of care assay systems, including Abbott's Point of Care (i-STAT™) electrochemical immunoassay system. Immunosensors and methods of manufacturing and operating them in single-use test devices are described, for example in U.S. Pat. No. 5,063,081 and published US Patent Applications 20030170881, 20040018577, 20050054078, and 20060160164 (incorporated by reference herein for their teachings regarding same).

By way of example, and not of limitation, examples of the present invention shall now be provided.

### EXAMPLE 1

### Acridinium-9-carboxamide Labeling Reagents

a) Acridinium, 9-[[[(4-methylphenyl)sulfonyl](3-sulfopropyl)amino]carbonyl]-10-[10-oxo-10-(pentafluorophenoxy)decyl]-, inner salt (Compound 1, shown below) was prepared according to the procedure described in Adamczyk, M.; Mattingly, P. G.; Moore, J. A.; Pan, Y. Org. Lett., 5, 3779-3782 (2003). The extinction coefficient for this label at 369 nm was 12,300 M⁻¹ cm⁻¹.

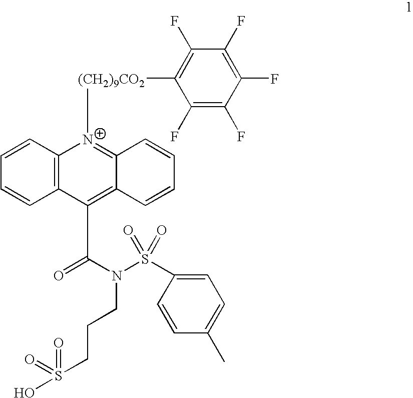

b) Acridinium, 9-[[[4-[(2,5-dioxo-1-pyrrolidinyl)oxy]-4-oxobutyl][(4-methylphenyl)sulfonyl]amino]carbonyl]-10-(3-sulfopropyl), inner salt (Compound 2, shown below) was prepared according to the procedure described in Adamczyk, M.; Chen, Y.-Y.; Mattingly, P. G.; Pan, Y. J. Org. Chem., 63, 5636-5639 (1998).

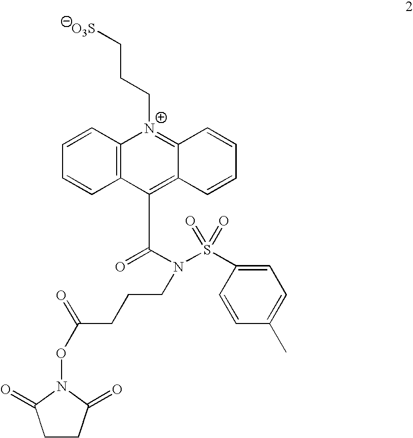

c) Preparation of acridinium, 9-[[[[4-[4-pentafluorophenoxy]-4-oxobutyl]phenyl]sulfonyl](3-sulfopropyl)amino]carbonyl]-10-(3-sulfopropyl), inner salt (Compound 3, shown below). Acridinium, 9-[[[[4-(3-carboxypropyl)phenyl]sulfonyl](3-sulfopropyl)amino]carbonyl]-10-(3-sulfopropyl), inner salt (700 mg, 1 mmol) (Adamczyk, M.; Chen, Y.-Y.; Mattingly, P. G.; Pan, Y. J. Org. Chem., 63, 5636-5639 (1998)) was dissolved in N,N-dimethylformamide (4 mL). Pyridine (0.81 mL, 10 mmol) and pentafluorophenyl trifluoroacetate (1.8 mL, 10 mmol) were added. The reaction mixture was stirred for 4 hours in the dark at ambient temperature. Afterwards, the solvent was removed in vacuo and the residue was triturated with diethyl ether (2×20 mL). Residual solvent was removed in vacuo. The crude pentafluorophenyl active ester was dissolved in acetonitrile (5 mL) then purified batch-wise (5×1 mL) by preparative reversed-phase HPLC on a ODS AQ 47×300, 15μ, 120A, column (YMC, Inc., Kyoto, JP, Cat No. AQ12S 153047RC) eluting at 75 mL/minute with an 11 minute gradient starting with 80:20 0.05% aqueous trifluoroacetic acid/acetonitrile and ending with 40:60 0.05% aqueous trifluoroacetic acid/acetonitrile. The combined fractions containing the pentafluorophenyl active ester were evaporated in vacuo at ambient temperature to remove the volatile organic solvent, and the remaining aq solution was then lyophilized. The residue was mixed with acetonitrile (14 mL) then evaporated in vacuo at 35-40° C., twice. Finally, the residue was transferred to a centrifuge tube with acetonitrile (14 mL) and the purified pentafluorophenyl active ester was collected after centrifugation at 5000 g and dried in vacuo at 45-50° C. The yield was 500 mg. Purity>95%. ε369 (14,900 M⁻¹ cm⁻¹).

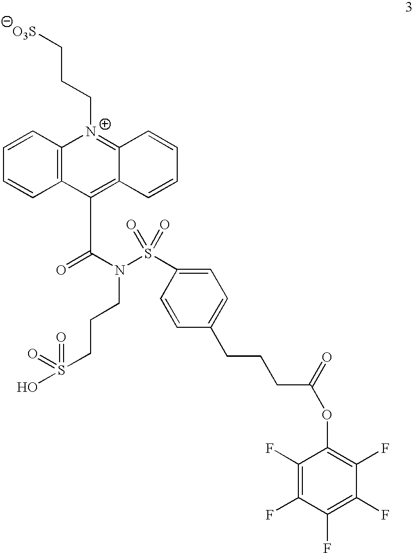

d) Acridinium, 9-[[[[4-[4-[(2,5-dioxo-1-pyrrolidinyl)oxy]-4-oxobutyl]phenyl]sulfonyl](3-sulfopropyl)amino]carbonyl]-10-(3-sulfopropyl), inner salt (Compound 4, shown below) was prepared according to the procedure described in Adamczyk, M.; Mattingly, P. G.; Moore, J. A.; Pan, Y; Shreder, K; Yu, Z. Bioconjugate Chem., 12, 329-331 (2001).

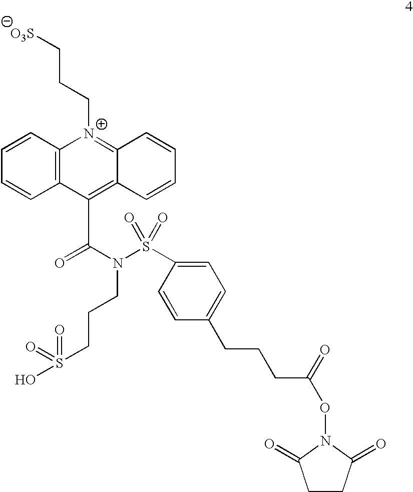

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method of detecting an iron metalloprotein in a test sample, the method comprising the steps of:
a) adding an acridinium-9-carboxamide-antibody conjugate to the test sample, wherein the antibody specifically binds the iron metalloprotein;
b) generating in or providing to the test sample a source of hydrogen peroxide before or after the addition of an acridinium-9-carboxamide-antibody conjugate;
c) adding a basic solution to the test sample to generate a light signal; and
d) quantifying the light generated to detect the iron metalloprotein.

2. The method of claim 1, wherein the iron metalloprotein is selected from the group consisting of: myeloperoxidase, ferritin, transferrin, lactoperoxidase, lactoferrin, feffedoxin, frataxin, divalent metal transporter 1, myoinositol oxygenase, rubrerythrin, thyroid peroxidase, methemoglobin and hemoglobin.

3. The method of claim 1, wherein the antibody is selected from the group consisting of: a polyclonal antibody, a monoclonal antibody, a chimeric antibody, a human antibody, a humanized antibody, a recombinant antibody, a single-chain Fv, an affinity maturated antibody, a single chain antibody, a single domain antibody, a Fab fragment, a F(ab′) fragment, a disulfide-linked Fv, an anti-idiotypic antibody and a functionally active epitope-binding fragment of any of the above.

4. The method of claim 1, wherein the test sample is whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid or semen.

5. The method of claim 1, wherein the acridinium-9-carboxamide-antibody conjugate is prepared from an acridinium-9-carboxamide having a structure according to formula I: 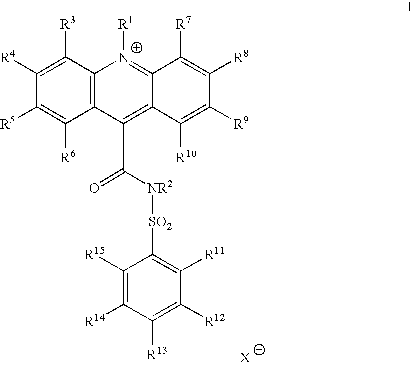
wherein R¹ and R² are each independently selected from the group consisting of: alkyl, alkenyl, alkynyl, aryl or aralkyl, sulfoalkyl, carboxyalkyl and oxoalkyl, and
wherein R³ through R¹³ are each independently selected from the group consisting of: hydrogen, alkyl, alkenyl, alkynyl, aryl or aralkyl, amino, amido, acyl, alkoxyl,
hydroxyl, carboxyl, halogen, halide, nitro, cyano, sulfo, sulfoalkyl, carboxyalkyl and oxoalkyl; and
optionally, if present, X^{⊖} is an anion.

6. The method of claim 1, wherein the hydrogen peroxide is provided by adding a buffer or a solution containing hydrogen peroxide.

7. The method of claim 1, wherein the hydrogen peroxide is generated by adding a hydrogen peroxide generating enzyme to the test sample.

8. The method of claim 1, further comprising quantifying the amount of iron metalloprotein in the test sample by relating the amount of light generated in the test sample by comparison to a standard curve for said iron metalloprotein.

9. The method of claim 9, wherein the standard curve is generated from solutions of an iron metalloprotein of a known concentration.

10. A kit for use in detecting an iron metalloprotein in a test sample, the kit comprising:
a. at least one acridinium-9-carboxamide;
b. at least one antibody that specifically binds the iron metalloprotein;
c. at least one basic solution;
d. a source of hydrogen peroxide; and
e. instructions for detecting an iron metalloprotein in a test sample.

11. The kit of claim 10, wherein the acridinium-9-carboxamide has a structure according to formula I: 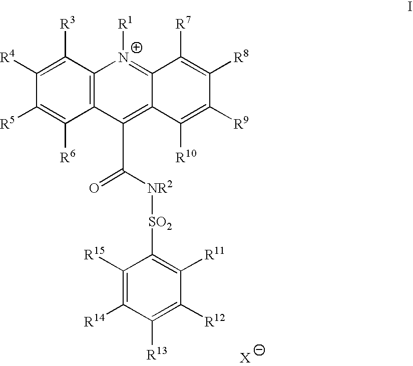
wherein R¹ and R² are each independently selected from the group consisting of: alkyl, alkenyl, alkynyl, aryl or aralkyl, sulfoalkyl, carboxyalkyl and oxoalkyl, and
wherein R³ through R¹⁵ are each independently selected from the group consisting of: hydrogen, alkyl, alkenyl, alkynyl, aryl or aralkyl, amino, amido, acyl, alkoxyl,
hydroxyl, carboxyl, halogen, halide, nitro, cyano, sulfo, sulfoalkyl, carboxyalkyl and oxoalkyl; and
optionally, if present, X^{⊖} is an anion.

12. The kit of claim 10, wherein the source of hydrogen peroxide is a buffer or a solution containing hydrogen peroxide.

13. A kit for use in detecting an iron metalloprotein in a test sample, the kit comprising:
a. at least one acridinium-9-carboxamide;
b. at least one antibody that specifically binds the iron metalloprotein;
c. at least one basic solution;
d. a means of generating hydrogen peroxide in situ in the test sample; and
e. instructions for detecting an iron metalloprotein in a test sample.

14. The kit of claim 13, wherein the acridinium-9-carboxamide has a structure according to formula I: 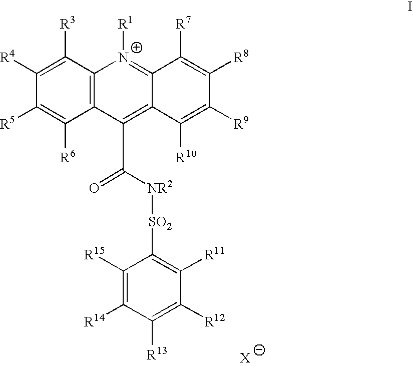
wherein R¹ and R² are each independently selected from the group consisting of: alkyl, alkenyl, alkynyl, aryl or aralkyl, sulfoalkyl, carboxyalkyl and oxoalkyl, and
wherein R³ through R¹⁵ are each independently selected from the group consisting of: hydrogen, alkyl, alkenyl, alkynyl, aryl or aralkyl, amino, amido, acyl, alkoxyl,
hydroxyl, carboxyl, halogen, halide, nitro, cyano, sulfo, sulfoalkyl, carboxyalkyl and oxoalkyl; and
optionally, if present, X^{⊖} is an anion.

15. The kit of claim 13, wherein said kit further comprises instructions for generating hydrogen peroxide in situ in the test sample.

16. The kit of claim 13, wherein the means for generating hydrogen peroxide in situ is a hydrogen peroxide generating enzyme.

17. The kit of claim 16, wherein the hydrogen peroxide generating enzyme is selected from the group consisting of: (R)-6-hydroxynicotine oxidase, (S)-2-hydroxy acid oxidase, (S)-6-hydroxynicotine oxidase, 3-aci-nitropropanoate oxidase, 3-hydroxyanthranilate oxidase, 4-hydroxymandelate oxidase, 6-hydroxynicotinate dehydrogenase, abscisic-aldehyde oxidase, acyl-CoA oxidase, alcohol oxidase, aldehyde oxidase, amine oxidase, amine oxidase (copper-containing), amine oxidase (flavin-containing), aryl-alcohol oxidase, aryl-aldehyde oxidase, catechol oxidase, cholesterol oxidase, choline oxidase, columbamine oxidase, cyclohexylamine oxidase, cytochrome c oxidase, D-amino-acid oxidase, D-arabinono-1,4-lactone oxidase, D-arabinono-1,4-lactone oxidase, D-aspartate oxidase, D-glutamate oxidase, D-glutamate(D-aspartate) oxidase, dihydrobenzophenanthridine oxidase, dihydroorotate oxidase, dihydrouracil oxidase, dimethylglycine oxidase, D-mannitol oxidase, ecdysone oxidase, ethanolamine oxidase, galactose oxidase, glucose oxidase, glutathione oxidase, glycerol-3-phosphate oxidase, glycine oxidase, glyoxylate oxidase, hexose oxidase, hydroxyphytanate oxidase, indole-3-acetaldehyde oxidase, lactic acid oxidase, L-amino-acid oxidase, L-aspartate oxidase, L-galactonolactone oxidase, L-glutamate oxidase, L-gulonolactone oxidase, L-lysine 6-oxidase, L-lysine oxidase, long-chain-alcohol oxidase, L-pipecolate oxidase, L-sorbose oxidase, malate oxidase, methanethiol oxidase, monoamino acid oxidase, N6-methyl-lysine oxidase, N-acylhexosamine oxidase, NAD(P)H oxidase, nitroalkane oxidase, N-methyl-L-amino-acid oxidase, nucleoside oxidase, oxalate oxidase, polyamine oxidase, polyphenol oxidase, polyvinyl-alcohol oxidase, prenylcysteine oxidase, protein-lysine 6-oxidase, putrescine oxidase, pyranose oxidase, pyridoxal 5′-phosphate synthase, pyridoxine 4-oxidase, pyrroloquinoline-quinone synthase, pyruvate oxidase, pyruvate oxidase (CoA-acetylating), reticuline oxidase, retinal oxidase, rifamycin-B oxidase, sarcosine oxidase, secondary-alcohol oxidase, sulfite oxidase, superoxide dismutase, superoxide reductase, tetrahydroberberine oxidase, thiamine oxidase, tryptophan α,β-oxidase, urate oxidase (uricase, uric acid oxidase), vanillyl-alcohol oxidase, xanthine oxidase, xylitol oxidase and combinations thereof.
